(19) United States
(12) Patent Application Publication
(10) Pub. No.: US 2024/0116979 A1
(43) Pub. Date: Apr. 11, 2024

(54) METHOD FOR PRODUCING LIPIDIC PEPTIDE

(71) Applicant: NISSAN CHEMICAL CORPORATION, Tokyo (JP)

(72) Inventors: Takeaki SHOJI, Funabashi-shi (JP); Hiroki YAMAGUCHI, Funabashi-shi (JP)

(73) Assignee: NISSAN CHEMICAL CORPORATION, Tokyo (JP)

(21) Appl. No.: 18/269,716

(22) PCT Filed: Dec. 24, 2021
(86) PCT No.: PCT/JP2021/048380

§ 371 Date: Jul. 3, 2023

(30) Foreign Application Priority Data
Dec. 25, 2020 (JP) 2020-217822

(51) Int. Cl. C07K 1/02

(52) U.S. Cl. CPC C07K 1/02

(57) ABSTRACT

[Task] There is provided a practical method for producing a lipidic peptide compound, the method enabling to mass-produce the lipidic peptide compound inexpensively with no need of complicated operations.

[Solution] There is provided a method to mix a non-polar solvent solution of an ester compound represented by formula (1)

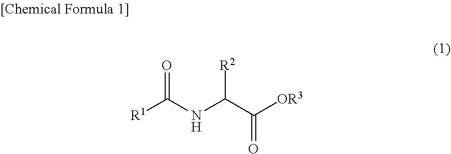

with a non-polar organic solvent containing an α-amino acid compound and a base represented by formula (2)

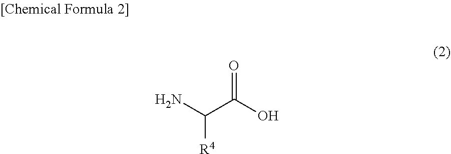

so as to produce a lipidic peptide compound or a pharmacologically acceptable salt thereof represented by formula (3).

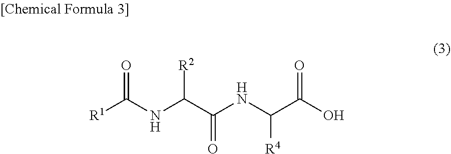

### TECHNICAL FIELD

The present invention relates to a method for producing a lipidic peptide.

### BACKGROUND ART

In recent years, a proposal has been made to use, as a hydrogelator, a novel lipidic peptide (lipidic peptide compound) prepared by binding of glycine or histidine to palmitic acid, etc., and a method of supplying the lipidic peptide has become important (Patent Document 1).

Meanwhile, a method involving solid-phase peptide synthesis has been generally shown as a production method for a lipidic peptide. However, the method can only be used for small-scale synthesis, and is difficult to apply to large-scale production.

Hitherto, the present inventors have reported that a lipidic peptide compound can be directly produced through reaction (amidation) between an amino group of an amino acid and an ester compound in the presence of a base in a solvent containing a non-polar organic solvent without use of a protective group (Patent Document 2).

### PRIOR ART DOCUMENTS

### Patent Documents

Patent Document 1: WO 2010/013555
Patent Document 2: WO 2011/027897

### SUMMARY OF THE INVENTION

### Problems to be Solved by the Invention

An object of the present invention is to provide a practical production method for a lipidic peptide compound, wherein the method does not require complicated operations and can mass-produce a lipidic peptide compound at low cost, as compared with conventional production methods.

### Means for Solving the Problems

In order to achieve the aforementioned object, the present inventors have conducted extensive studies, and as a result have found that a lipidic peptide compound can be directly produced through reaction (amidation) between an amino group of an amino acid and an ester compound in the presence of a base in a solvent containing a non-polar organic solvent without use of a protective group, and that the lipidic peptide is produced at high yield by mixing a preliminarily prepared solution containing the amino acid and the base with a solution of the ester. Also, the present inventors have found that isolation of the resultant lipidic peptide compound at the isoelectric point leads to an improvement in operability. The present invention has been accomplished on the basis of these findings.

Accordingly, the present invention is directed to the followings.

[1] A production method for a lipidic peptide compound of the following Formula (3):

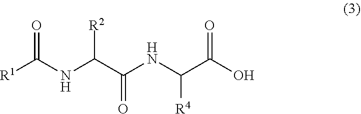

(wherein R¹, R², and R⁴ have the meanings as defined below) or a pharmaceutically usable salt thereof, the production method being characterized by comprising:
a mixing step of mixing a non-polar solvent containing an ester compound of the following Formula (1):

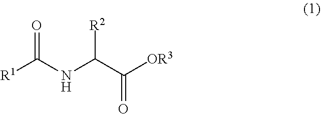

(wherein R¹ is a C₉₋₂₃ aliphatic group; R² is a hydrogen atom or a C₁₋₄ alkyl group possibly having a branched chain having a carbon atom number of 1 or 2; and R³ is a C₁₋₆ alkyl group, a C₁₋₆ haloalkyl group, a C₁₋₆ hydroxyalkyl group, or an aryl group substitutable with a C₁₋₆ alkyl group) with a non-polar organic solvent containing an α-amino acid compound of the following Formula (2):

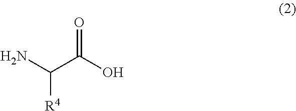

(wherein R⁴ is a —(CH₂)ₙ—X group; n is a number of 1 to 4; and X is an amino group, a guanidino group, a —CONH₂ group, or a 5-membered or 6-membered ring or condensed heterocyclic ring composed of 5-membered and 6-membered rings possibly having one to three nitrogen atoms) and a base.

[2] The production method according to [1], characterized in that the non-polar solvent contains a non-polar organic solvent and an alcohol.

[3] The production method according to [1], wherein, in the aforementioned formula, n is a number of 1 to 4, and X is an amino group, a guanidino group, or a —CONH₂ group; or n is 1, and X is a pyrrole group, an imidazole group, a pyrazole group, or an imidazole group.

[4] The production method according to [1], wherein, in the aforementioned formula, R¹ is a Cu-21 aliphatic group having a linear or branched structure and possibly having zero to two unsaturated bonds.

[5] The production method according to [1], wherein, in the aforementioned formula, R² is a hydrogen atom or a C₁₋₃ alkyl group possibly having a branched chain having a carbon atom number of 1.

[6] The production method according to [1], wherein, in the aforementioned formula, R² is a hydrogen atom, a methyl group, an ethyl group, an n-propyl group, an isopropyl group, an n-butyl group, an isobutyl group, a sec-butyl group, or a tert-butyl group, and R⁴ is an aminomethyl group, an aminoethyl group, a 3-aminopropyl group, a 4-aminobutyl group, a carbamoylmethyl group, a 2-carbamoylethyl group, a 3-carbamoylbutyl group, a 2-guanidinoethyl group, a 3-guanidinopropyl group, a pyrrolemethyl group, an imidazolemethyl group, a pyrazolemethyl group, or a 3-indolemethyl group.

[7] The production method according to [6], wherein, in the aforementioned formula, R² is a hydrogen atom, a methyl group, an isopropyl group, an isobutyl group, or a sec-butyl group, and R⁴ is a 4-aminobutyl group, a carbamoylmethyl group, a 2-carbamoylethyl group, a 3-guanidinopropyl group, an imidazolemethyl group, or a 3-indolemethyl group.

[8] The production method according to [1], wherein, in the aforementioned formula, R³ is a methyl group or an ethyl group.

[9] The production method according to any one of [1] to [8], wherein the base is at least one selected from among an alkali metal, an alkali metal inorganic acid salt, an alkali metal hydroxide, an alkali metal alkoxide, an alicyclic amine, or an alcohol solution of any of these, or an alcohol dispersion of any of these.

[10] The production method according to [9], wherein the base is at least one selected from among metallic sodium, metallic potassium, sodium carbonate, potassium carbonate, potassium phosphate, sodium phosphate, sodium hydroxide, potassium hydroxide, sodium methoxide, sodium ethoxide, potassium methoxide, potassium ethoxide, t-butoxypotassium, 1,8-diazabicyclo[5.4.0]-7-undecene, 1,5-diazabicyclo[4.3.0]-5-nonene, or an alcohol solution of any of these, or an alcohol dispersion of any of these.

[11] The production method according to [10], wherein the base is sodium methoxide, or a methanol solution thereof, or a methanol dispersion thereof.

[12] The production method according to any one of [1] to [11], wherein the non-polar organic solvent is at least one selected from the group consisting of an aromatic compound, a saturated aliphatic compound, and an unsaturated aliphatic compound.

[13] The production method according to [12], wherein the non-polar organic solvent is at least one selected from the group consisting of toluene, xylene, o-dichlorobenzene, pentane, hexane, heptane, octane, cyclopentane, cyclohexane, methylcyclohexane, cycloheptane, and 1-hexene.

[14] The production method according to [2], wherein the non-polar solvent contains toluene and methanol or ethanol.

[15] The production method according to any one of [1] to [14], wherein the reaction between the ester compound of Formula (1) and the α-amino acid compound of Formula (2) is performed at a reaction temperature of 70±5° C.

[16] The production method according to any one of [1] to [15], wherein the method comprises a precipitation step of treating a product produced through the reaction between the ester compound of Formula (1) and the α-amino acid compound of Formula (2) with a hydrogen halide so that the pH of the product is adjusted to the isoelectric point of the lipidic peptide compound, thereby precipitating the lipidic peptide.

[17] The production method according to [16], wherein the precipitation step is performed at 40° C. to 70° C.

[18] A production method for a lipidic peptide compound of the following Formula (3):

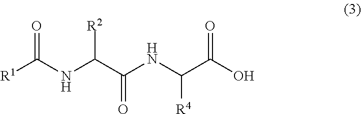

(wherein R¹, R², and R⁴ have the meanings as defined below) or a pharmaceutically usable salt thereof, the production method being characterized by comprising: a step of reacting a compound of the following Formula (4):

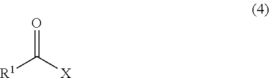

(wherein X is a halogen atom, a C₁₋₆ alkoxy group, or a —OC(O)R¹ group; and R¹ is a C₉₋₂₃ aliphatic group) with a compound of the following Formula (5):

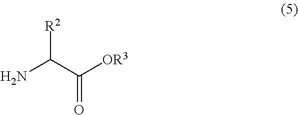

(wherein R² is a hydrogen atom or a C₁₋₄ alkyl group possibly having a branched chain having a carbon atom number of 1 or 2; and R³ is a C₁₋₆ alkyl group, a C₁₋₆ haloalkyl group, a C₁₋₆ hydroxyalkyl group, or an aryl group substitutable with a C₁₋₆ alkyl group), thereby producing an ester compound of the following Formula (1):

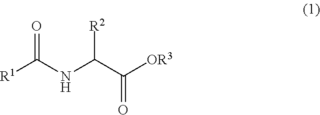

(wherein R¹, R², and R³ have the same meanings as defined above); and a mixing step of mixing a non-polar solvent containing the ester compound of Formula (1) with a non-polar organic solvent containing an α-amino acid compound of the following Formula (2):

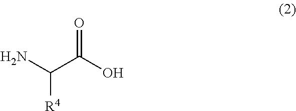

(wherein R⁴ is a hydrogen atom, a C₁₋₇ alkyl group possibly having a branched chain having a carbon atom number of 1 to 3, a phenylmethyl group, a phenylethyl group, or a —(CH₂)ₙ—X group; n is a number of 1 to 4; and X is an amino group, a guanidino group, a —CONH₂ group, or a 5-membered or 6-membered ring or condensed heterocyclic ring composed of 5-membered and 6-membered rings possibly having one to three nitrogen atoms) and a base.

### Effects of the Invention

The production method of the present invention can produce a desired lipidic peptide compound at high yield.

The production method of the present invention is a practical production method applicable to industrial production, since the method involves neither racemization of an amino acid to be used nor complicated protection and deprotection operations, and involves no use of an expensive reagent such as a condensing agent.

Furthermore, the present invention can be applied to the case where a target lipidic peptide compound has a gelation ability and thus encounters difficulty in isolating a free form.

### MODES FOR CARRYING OUT THE INVENTION

As described above, various methods have been proposed for producing a lipidic peptide compound. However, demand has arisen for a method capable of mass-producing a lipidic peptide compound without requiring complicated operations such as protection and deprotection of functional groups or expensive condensing agents and protective group reagents.

In view of the foregoing, the present inventors have conceived a method in which R³ is used as a protective group for the production of an ester compound of Formula (1) to thereby improve the yield of a product and operability, and the thus-formed —OR³ moiety is used as a leaving group in subsequent amidation between the ester compound and an α-amino acid compound of Formula (2). The inventors have found that this method serves as an economical, low-waste, and environmentally friendly production method.

Furthermore, the method involves the use of a non-polar solvent and an alcohol as reaction solvents, the reaction in an almost homogeneous mixed solvent under heating conditions, and precipitation of a product; i.e., a lipidic peptide compound salt (e.g., alkali metal salt) after completion of the reaction. The inventors have enabled efficient production of a salt of the product through filtration of the precipitate.

The use of a non-polar solvent can prevent gelation after completion of the reaction and cooling, unlike a conventional case where DMF or water is used for the production of a lipidic peptide compound. In addition, the use of an aqueous hydrogen chloride solution in an amount necessary for neutralization of the liquid (alkaline liquid) obtained through the reaction results in completion of the neutralization without causing gelation, and enables easy recovery of a free form.

Thus, the present inventors have found that a lipidic peptide compound can be simply synthesized at high yield without causing racemization of the amino acid used. The present invention has been accomplished on the basis of this finding.

The present invention will next be described in more detail.

In the present specification, “n” denotes normal; “i” denotes iso; “s” or “sec” denotes secondary; “t” or “tert” denotes tertiary; “c” denotes cyclo; “o” denotes ortho; “m” denotes meta; “p” denotes para; “Me” denotes a methyl group; “Bu” denotes a butyl group; and “tBu” denotes a tertiary butyl group.

In Formula (1), R¹ is a C₉₋₂₃ aliphatic group. Preferably, R¹ is a Cu-21 aliphatic group having a linear or branched structure, or a linear aliphatic group having a carbon atom number of 11 to 21 and having one or two unsaturated bonds.

Particularly preferred specific examples of the aliphatic group of R¹ include nonyl group, decyl group, undecyl group, dodecyl group (lauryl group), tridecyl group, tetradecyl group (myristyl group), pentadecyl group, hexadecyl group (palmityl group), heptadecyl group (margaryl group), octadecyl group (stearyl group), nonadecyl group, icosyl group, and henicosyl group.

In Formula (1), R² is a hydrogen atom or a C₁₋₄ alkyl group possibly having a branched chain having a carbon atom number of 1 or 2.

The “C₁₋₄ alkyl group possibly having a branched chain having a carbon atom number of 1 or 2” of R² refers to an alkyl group having a main chain having a carbon atom number of 1 to 4 and possibly having a branched chain having a carbon atom number of 1 or 2. Specific examples of the alkyl group include methyl group, ethyl group, n-propyl group, i-propyl group, n-butyl group, i-butyl group, sec-butyl group, or tert-butyl group.

R² is preferably a hydrogen atom or a C₁₋₃ alkyl group possibly having a branched chain having a carbon atom number of 1, more preferably a hydrogen atom. The “C₁₋₃ alkyl group possibly having a branched chain having a carbon atom number of 1” refers to an alkyl group having a main chain having a carbon atom number of 1 to 3 and possibly having a branched chain having a carbon atom number of 1. Specific examples of the alkyl group include methyl group, ethyl group, n-propyl group, i-propyl group, i-butyl group, or sec-butyl group. Preferred is a methyl group, an i-propyl group, an i-butyl group, or a sec-butyl group.

In Formula (1), R³ is a C₁₋₆ alkyl group, a C₁₋₆ haloalkyl group, a C₁₋₆ hydroxyalkyl group, or an aryl group substitutable with a C₁₋₆ alkyl group.

Particularly preferred specific examples of the alkyl group of R³ include methyl group, ethyl group, n-propyl group, i-propyl group, n-butyl group, i-butyl group, sec-butyl group, or tert-butyl group. More preferred is a methyl group or an ethyl group.

In Formula (2), R⁴ is a hydrogen atom, a C₁₋₇ alkyl group possibly having a branched chain having a carbon atom number of 1 to 3, a phenylmethyl group, a phenylethyl group, or a —(CH₂)ₙ—X group, and is preferably a —(CH₂)ₙ—X group. In the —(CH₂)ₙ—X group, n is a number of 1 to 4, and X is an amino group, a guanidino group, a —CONH₂ group, or a 5-membered or 6-membered ring or condensed heterocyclic ring composed of 5-membered and 6-membered rings possibly having one to three nitrogen atoms.

In the —(CH₂)ₙ—X group, X is preferably an amino group, a guanidino group, a —CONH₂ group, a pyrrole group, an imidazole group, a pyrazole group, or an indole group, more preferably an imidazole group. In the —(CH₂)ₙ— group, n is preferably 1 or 2, more preferably 1.

Thus, the —(CH₂)ₙ— group is preferably an aminomethyl group, a 2-aminoethyl group, a 3-aminopropyl group, a 4-aminobutyl group, a carbamoylmethyl group, a 2-carbamoylethyl group, a 3-carbamoylbutyl group, a 2-guanidinoethyl group, a 3-guanidinopropyl group, a pyrrolemethyl group, an imidazolemethyl group, a pyrazolemethyl group, or a 3-indolemethyl group, more preferably a 4-aminobutyl group, a carbamoylmethyl group, a 2-carbamoylethyl group, a 3-guanidinopropyl group, an imidazolemethyl group, or a 3-indolemethyl group, still more preferably an imidazolemethyl group.

Therefore, specific examples of particularly suitable lipidic peptide compounds of Formula (3) are the following compounds each being formed of a lipidic moiety and a dipeptide moiety. The amino acid abbreviations used are histidine (His), glycine (Gly), valine (Val), isoleucine (Ile), alanine (Ala), arginine (Arg), asparagine (Asn), glutamine (Gln), leucine (Leu), lysine (Lys), and tryptophan (Trp).

Specific examples of the compounds include N-lauroyl-Gly-His, N-lauroyl-Gly-Trp, N-lauroyl-Gly-Gln, N-lauroyl-Gly-Asn, N-lauroyl-Gly-Arg, N-lauroyl-Gly-Lys, N-lauroyl-Ala-His, N-lauroyl-Ala-Trp, N-lauroyl-Ala-Gln, N-lauroyl-Ala-Asn, N-lauroyl-Ala-Arg, N-lauroyl-Ala-Lys, N-lauroyl-Val-His, N-lauroyl-Val-Trp, N-lauroyl-Val-Gln, N-lauroyl-Val-Asn, N-lauroyl-Val-Arg, N-lauroyl-Val-Lys, N-lauroyl-Leu-His, N-lauroyl-Leu-Trp, N-lauroyl-Leu-Gln, N-lauroyl-Leu-Asn, N-lauroyl-Leu-Arg, N-lauroyl-Leu-Lys, N-lauroyl-Ile-His, N-lauroyl-Ile-Trp, N-lauroyl-Ile-Gln, N-lauroyl-Ile-Asn, N-lauroyl-Ile-Arg, N-lauroyl-Ile-Lys, N-myristoyl-Gly-His, N-myristoyl-Gly-Trp, N-myristoyl-Gly-Gln, N-myristoyl-Gly-Asn, N-myristoyl-Gly-Arg, N-myristoyl-Gly-Lys, N-myristoyl-Ala-His, N-myristoyl-Ala-Trp, N-myristoyl-Ala-Gln, N-myristoyl-Ala-Asn, N-myristoyl-Ala-Arg, N-myristoyl-Ala-Lys, N-myristoyl-Val-His, N-myristoyl-Val-Trp, N-myristoyl-Val-Gln, N-myristoyl-Val-Asn, N-myristoyl-Val-Arg, N-myristoyl-Val-Lys, N-myristoyl-Leu-His, N-myristoyl-Leu-Trp, N-myristoyl-Leu-Gln, N-myristoyl-Leu-Asn, N-myristoyl-Leu-Arg, N-myristoyl-Leu-Lys, N-myristoyl-Ile-His, N-myristoyl-Ile-Trp, N-myristoyl-Ile-Gln, N-myristoyl-Ile-Asn, N-myristoyl-Ile-Arg, N-myristoyl-Ile-Lys, N-palmitoyl-Gly-His, N-palmitoyl-Gly-Trp, N-palmitoyl-Gly-Gln, N-palmitoyl-Gly-Asn, N-palmitoyl-Gly-Arg, N-palmitoyl-Gly-Lys, N-palmitoyl-Ala-His, N-palmitoyl-Ala-Trp, N-palmitoyl-Ala-Gln, N-palmitoyl-Ala-Asn, N-palmitoyl-Ala-Arg, N-palmitoyl-Ala-Lys, N-palmitoyl-Val-His, N-palmitoyl-Val-Trp, N-palmitoyl-Val-Gln, N-palmitoyl-Val-Asn, N-palmitoyl-Val-Arg, N-palmitoyl-Val-Lys, N-palmitoyl-Leu-His, N-palmitoyl-Leu-Trp, N-palmitoyl-Leu-Gln, N-palmitoyl-Leu-Asn, N-palmitoyl-Leu-Arg, N-palmitoyl-Leu-Lys, N-palmitoyl-Ile-His, N-palmitoyl-Ile-Trp, N-palmitoyl-Ile-Gln, N-palmitoyl-Ile-Asn, N-palmitoyl-Ile-Arg, N-palmitoyl-Ile-Lys, N-margaroyl-Gly-His, N-margaroyl-Gly-Trp, N-margaroyl-Gly-Gln, N-margaroyl-Gly-Asn, N-margaroyl-Gly-Arg, N-margaroyl-Gly-Lys, N-margaroyl-Ala-His, N-margaroyl-Ala-Trp, N-margaroyl-Ala-Gln, N-margaroyl-Ala-Asn, N-margaroyl-Ala-Arg, N-margaroyl-Ala-Lys, N-margaroyl-Val-His, N-margaroyl-Val-Trp, N-margaroyl-Val-Gln, N-margaroyl-Val-Asn, N-margaroyl-Val-Arg, N-margaroyl-Val-Lys, N-margaroyl-Leu-His, N-margaroyl-Leu-Trp, N-margaroyl-Leu-Gln, N-margaroyl-Leu-Asn, N-margaroyl-Leu-Arg, N-margaroyl-Leu-Lys, N-margaroyl-Ile-His, N-margaroyl-Ile-Trp, N-margaroyl-Ile-Gln, N-margaroyl-Ile-Asn, N-margaroyl-Ile-Arg, N-margaroyl-Ile-Lys, N-margaroyl-Gly-His, N-margaroyl-Gly-Trp, N-margaroyl-Gly-Gln, N-margaroyl-Gly-Asn, N-margaroyl-Gly-Arg, N-margaroyl-Gly-Lys, N-margaroyl-Ala-His, N-margaroyl-Ala-Trp, N-margaroyl-Ala-Gln, N-margaroyl-Ala-Asn, N-margaroyl-Ala-Arg, N-margaroyl-Ala-Lys, N-margaroyl-Val-His, N-margaroyl-Val-Trp, N-margaroyl-Val-Gln, N-margaroyl-Val-Asn, N-margaroyl-Val-Arg, N-margaroyl-Val-Lys, N-margaroyl-leu-His, N-margaroyl-Leu-Trp, N-margaroyl-Leu-Gln, N-margaroyl-Leu-Asn, N-margaroyl-Leu-Arg, N-margaroyl-Leu-Lys, N-margaroyl-Ile-His, N-margaroyl-Ile-Trp, N-margaroyl-Ile-Gln, N-margaroyl-Ile-Asn, N-margaroyl-Ile-Arg, N-margaroyl-Ile-Lys, N-stearoyl-Gly-His, N-stearoyl-Gly-Trp, N-stearoyl-Gly-Gln, N-stearoyl-Gly-Asn, N-stearoyl-Gly-Arg, N-stearoyl-Gly-Lys, N-stearoyl-Ala-His, N-stearoyl-Ala-Trp, N-stearoyl-Ala-Gln, N-stearoyl-Ala-Asn, N-stearoyl-Ala-Arg, N-stearoyl-Ala-Lys, N-stearoyl-Val-His, N-stearoyl-Val-Trp, N-stearoyl-Val-Gln, N-stearoyl-Val-Asn, N-stearoyl-Val-Arg, N-stearoyl-Val-Lys, N-stearoyl-Leu-His, N-stearoyl-Leu-Trp, N-stearoyl-Leu-Gln, N-stearoyl-Leu-Asn, N-stearoyl-Leu-Arg, N-stearoyl-Leu-Lys, N-stearoyl-Ile-His, N-stearoyl-Ile-Trp, N-stearoyl-Ile-Gln, N-stearoyl-Ile-Asn, N-stearoyl-Ile-Arg, N-stearoyl-Ile-Lys, N-elaidoyl-Gly-His, N-elaidoyl-Gly-Trp, N-elaidoyl-Gly-Gln, N-elaidoyl-Gly-Asn, N-elaidoyl-Gly-Arg, N-elaidoyl-Gly-Lys, N-elaidoyl-Ala-His, N-elaidoyl-Ala-Trp, N-elaidoyl-Ala-Gln, N-elaidoyl-Ala-Asn, N-elaidoyl-Ala-Arg, N-elaidoyl-Ala-Lys, N-elaidoyl-Val-His, N-elaidoyl-Val-Trp, N-elaidoyl-Val-Gln, N-elaidoyl-Val-Asn, N-elaidoyl-Val-Arg, N-elaidoyl-Val-Lys, N-elaidoyl-Leu-His, N-elaidoyl-Leu-Trp, N-elaidoyl-Leu-Gln, N-elaidoyl-Leu-Asn, N-elaidoyl-Leu-Arg, N-elaidoyl-Leu-Lys, N-elaidoyl-Ile-His, N-elaidoyl-Ile-Trp, N-elaidoyl-Ile-Gln, N-elaidoyl-Ile-Asn, N-elaidoyl-Ile-Arg, N-elaidoyl-Ile-Lys, N-arachidoyl-Gly-His, N-arachidoyl-Gly-Trp, N-arachidoyl-Gly-Gln, N-arachidoyl-Gly-Asn, N-arachidoyl-Gly-Arg, N-arachidoyl-Gly-Lys, N-arachidoyl-Ala-His, N-arachidoyl-Ala-Trp, N-arachidoyl-Ala-Gln, N-arachidoyl-Ala-Asn, N-arachidoyl-Ala-Arg, N-arachidoyl-Ala-Lys, N-arachidoyl-Val-His, N-arachidoyl-Val-Trp, N-arachidoyl-Val-Gln, N-arachidoyl-Val-Asn, N-arachidoyl-Val-Arg, N-arachidoyl-Val-Lys, N-arachidoyl-Leu-His, N-arachidoyl-Leu-Trp, N-arachidoyl-Leu-Gln, N-arachidoyl-Leu-Asn, N-arachidoyl-Leu-Arg, N-arachidoyl-Leu-Lys, N-arachidoyl-Ile-His, N-arachidoyl-Ile-Trp, N-arachidoyl-Ile-Gln, N-arachidoyl-Ile-Asn, N-arachidoyl-Ile-Arg, N-arachidoyl-Ile-Lys, N-behenoyl-Gly-His, N-behenoyl-Gly-Trp, N-behenoyl-Gly-Gln, N-behenoyl-Gly-Asn, N-behenoyl-Gly-Arg, N-behenoyl-Gly-Lys, N-behenoyl-Ala-His, N-behenoyl-Ala-Trp, N-behenoyl-Ala-Gln, N-behenoyl-Ala-Asn, N-behenoyl-Ala-Arg, N-behenoyl-Ala-Lys, N-behenoyl-Val-His, N-behenoyl-Val-Trp, N-behenoyl-Val-Gln, N-behenoyl-Val-Asn, N-behenoyl-Val-Arg, N-behenoyl-Val-Lys, N-behenoyl-Leu-His, N-behenoyl-Leu-Trp, N-behenoyl-Leu-Gln, N-behenoyl-Leu-Asn, N-behenoyl-Leu-Arg, N-behenoyl-Leu-Lys, N-behenoyl-Ile-His, N-behenoyl-Ile-Trp, N-behenoyl-Ile-Gln, N-behenoyl-Ile-Asn, N-behenoyl-Ile-Arg, and N-behenoyl-Ile-Lys.

Among the aforementioned compounds, more preferred lipidic peptide compounds are N-lauroyl-Gly-His, N-lauroyl-Gly-Trp, N-lauroyl-Gly-Gln, N-lauroyl-Gly-Asn, N-lauroyl-Gly-Lys, N-lauroyl-Ala-His, N-lauroyl-Ala-Trp, N-lauroyl-Ala-Gln, N-lauroyl-Ala-Asn, N-lauroyl-Ala-Lys, N-lauroyl-Val-His, N-lauroyl-Val-Trp, N-lauroyl-Val-Gln, N-lauroyl-Val-Asn, N-lauroyl-Val-Lys, N-myristoyl-Gly-His, N-myristoyl-Gly-Trp, N-myristoyl-Gly-Gln, N-myristoyl-Gly-Asn, N-myristoyl-Gly-Lys, N-myristoyl-Ala-His, N-myristoyl-Ala-Trp, N-myristoyl-Ala-Gln, N-myristoyl-Ala-Asn, N-myristoyl-Ala-Lys, N-myristoyl-Val-His, N-myristoyl-Val-Trp, N-myristoyl-Val-Gln, N-myristoyl-Val-Asn, N-myristoyl-Val-Lys, N-palmitoyl-Gly-His, N-palmitoyl-Gly-Trp, N-palmitoyl-Gly-Gln, N-palmitoyl-Gly-Asn, N-palmitoyl-Gly-Lys, N-palmitoyl-Ala-His, N-palmitoyl-Ala-Trp, N-palmitoyl-Ala-Gln, N-palmitoyl-Ala-Asn, N-palmitoyl-Ala-Lys, N-palmitoyl-Val-His, N-palmitoyl-Val-Trp, N-palmitoyl-Val-Gln, N-palmitoyl-Val-Asn, N-palmitoyl-Val-Lys, N-margaroyl-Gly-His, N-margaroyl-Gly-Trp, N-margaroyl-Gly-Gln, N-margaroyl-Gly-Asn, N-margaroyl-Gly-Lys, N-margaroyl-Ala-His, N-margaroyl-Ala-Trp, N-margaroyl-Ala-Gln, N-margaroyl-Ala-Asn, N-margaroyl-Ala-Lys, N-margaroyl-Val-His, N-margaroyl-Val-Trp, N-margaroyl-Val-Gln, N-margaroyl-Val-Asn, N-margaroyl-Val-Lys, N-margaroyl-Gly-His, N-margaroyl-Gly-Trp, N-margaroyl-Gly-Gln, N-margaroyl-Gly-Asn, N-margaroyl-Gly-Lys, N-margaroyl-Ala-His, N-margaroyl-Ala-Trp, N-margaroyl-Ala-Gln, N-margaroyl-Ala-Asn, N-margaroyl-Ala-Lys, N-margaroyl-Val-His, N-margaroyl-Val-Trp, N-margaroyl-Val-Gln, N-margaroyl-Val-Asn, N-margaroyl-Val-Lys, N-stearoyl-Gly-His, N-stearoyl-Gly-Trp, N-stearoyl-Gly-Gln, N-stearoyl-Gly-Asn, N-stearoyl-Gly-Lys, N-stearoyl-Ala-His, N-stearoyl-Ala-Trp, N-stearoyl-Ala-Gln, N-stearoyl-Ala-Asn, N-stearoyl-Ala-Lys, N-stearoyl-Val-His, N-stearoyl-Val-Trp, N-stearoyl-Val-Gln, N-stearoyl-Val-Asn, N-stearoyl-Val-Lys, N-elaidoyl-Gly-His, N-elaidoyl-Gly-Trp, N-elaidoyl-Gly-Gln, N-elaidoyl-Gly-Asn, N-elaidoyl-Gly-Lys, N-elaidoyl-Ala-His, N-elaidoyl-Ala-Trp, N-elaidoyl-Ala-Gln, N-elaidoyl-Ala-Asn, N-elaidoyl-Ala-Lys, N-elaidoyl-Val-His, N-elaidoyl-Val-Trp, N-elaidoyl-Val-Gln, N-elaidoyl-Val-Asn, N-elaidoyl-Val-Lys, N-arachidoyl-Gly-His, N-arachidoyl-Gly-Trp, N-arachidoyl-Gly-Gln, N-arachidoyl-Gly-Asn, N-arachidoyl-Gly-Lys, N-arachidoyl-Ala-His, N-arachidoyl-Ala-Trp, N-arachidoyl-Ala-Gln, N-arachidoyl-Ala-Asn, N-arachidoyl-Ala-Lys, N-arachidoyl-Val-His, N-arachidoyl-Val-Trp, N-arachidoyl-Val-Gln, N-arachidoyl-Val-Asn, N-arachidoyl-Val-Lys, N-behenoyl-Gly-His, N-behenoyl-Gly-Trp, N-behenoyl-Gly-Gln, N-behenoyl-Gly-Asn, N-behenoyl-Gly-Lys, N-behenoyl-Ala-His, N-behenoyl-Ala-Trp, N-behenoyl-Ala-Gln, N-behenoyl-Ala-Asn, N-behenoyl-Ala-Lys, N-behenoyl-Val-His, N-behenoyl-Val-Trp, N-behenoyl-Val-Gln, N-behenoyl-Val-Asn, and N-behenoyl-Val-Lys.

Most preferred compounds are N-lauroyl-Gly-His, N-lauroyl-Gly-Gln, N-lauroyl-Gly-Asn, N-lauroyl-Gly-Lys, N-myristoyl-Gly-His, N-myristoyl-Gly-Gln, N-myristoyl-Gly-Asn, N-myristoyl-Gly-Lys, N-palmitoyl-Gly-His, N-palmitoyl-Gly-Trp, N-palmitoyl-Gly-Gln, N-palmitoyl-Gly-Asn, N-palmitoyl-Gly-Lys, N-palmitoyl-Ala-His, N-palmitoyl-Ala-Trp, N-palmitoyl-Ala-Gln, N-palmitoyl-Ala-Asn, N-palmitoyl-Ala-Lys, N-palmitoyl-Val-His, N-palmitoyl-Val-Trp, N-palmitoyl-Val-Gln, N-palmitoyl-Val-Asn, N-palmitoyl-Val-Lys, N-margaroyl-Gly-His, N-margaroyl-Gly-Gln, N-margaroyl-Gly-Asn, N-margaroyl-Gly-Lys, N-margaroyl-Gly-His, N-margaroyl-Gly-Gln, N-margaroyl-Gly-Asn, N-margaroyl-Gly-Lys, N-stearoyl-Gly-His, N-stearoyl-Gly-Gln, N-stearoyl-Gly-Asn, N-stearoyl-Gly-Lys, N-elaidoyl-Gly-His, N-elaidoyl-Gly-Gln, N-elaidoyl-Gly-Asn, N-elaidoyl-Gly-Lys, N-arachidoyl-Gly-His, N-arachidoyl-Gly-Gln, N-arachidoyl-Gly-Asn, N-arachidoyl-Gly-Lys, N-behenoyl-Gly-His, N-behenoyl-Gly-Gln, N-behenoyl-Gly-Asn, and N-behenoyl-Gly-Lys. Examples of the lipidic peptide having a branched structure include N-2-(4,4-dimethylpentan-2-yl)-5,7,7-trimethyloctanoyl-Gly-His and N-2-heptylundecanoyl-Gly-His.

No particular limitation is imposed on the base used for the reaction between the ester compound of Formula (1) and the α-amino acid compound of Formula (2). Examples of the base include alkali metals such as metallic sodium and metallic potassium; alkali metal inorganic acid salts such as sodium carbonate, potassium carbonate, potassium phosphate, and sodium phosphate; alkali metal hydroxides such as sodium hydroxide and potassium hydroxide; alkali metal alkoxides such as sodium methoxide and t-butoxypotassium; aliphatic amines such as triethylamine and tri-n-butylamine; alicyclic amines such as 1,8-diazabicyclo[5.4.0]-7-undecene (hereinafter may be referred to as “DBU”) and 1,5-diazabicyclo[4.3.0]-5-nonene (hereinafter may be referred to as “DBN”); aromatic amines such as pyridine and 2-methyl-5-ethylpyridine; and alcohol solutions or alcohol dispersions of these base (solid) compounds. These bases may be used alone or in combination of two or more species.

Among the aforementioned bases, preferred is sodium methoxide, sodium ethoxide, potassium methoxide, potassium ethoxide, t-butoxypotassium, DBU, or DBN, and preferred is sodium methoxide, or an alcohol solution or alcohol dispersion of such a metal alkoxide, in view of increasing percent conversion to thereby further improve the yield of a target product.

Sodium methoxide may be in the form of solid, methanol solution, or methanol dispersion. Alternatively, the sodium methoxide used may be prepared preliminarily or in the reaction system by using metallic sodium and methanol. In consideration of operability and yield, commercially available about 28% sodium methoxide methanol solution is preferably used.

No particular limitation is imposed on the amount of the base used, and the amount is generally about 1 equivalent to 10 equivalents, preferably 1 equivalent to 5 equivalents, more preferably 1.3 equivalents to 2 equivalents, relative to the compound of Formula (1).

No particular limitation is imposed on the non-polar organic solvent contained in the solvent used for the aforementioned reaction, and a non-polar organic solvent that does not affect the reaction may be appropriately selected from various solvents used for general organic synthesis.

Specific examples of the non-polar organic solvent include saturated aliphatic hydrocarbon compounds such as pentane, c-pentane, hexane, c-hexane, methyl-c-hexane, heptane, c-heptane, octane, decane, and decalin; unsaturated aliphatic hydrocarbon compounds such as 1-hexene and 1-octyne; and aromatic hydrocarbon compounds such as benzene, toluene, xylene, and o-dichlorobenzene. These solvents may be used alone or in combination of two or more species.

Among these non-polar organic solvents, preferred is at least one selected from the group consisting of toluene, xylene, o-dichlorobenzene, pentane, hexane, heptane, octane, c-pentane, c-hexane, methyl-c-hexane, c-heptane, and 1-hexene, and particularly preferred is toluene, in view of preventing the hydrolysis of the ester compound of Formula (1), and increasing percent conversion to thereby further improve the yield of a target product.

The solvent used for the aforementioned reaction preferably contains an alcohol in addition to the aforementioned non-polar solvent. No particular limitation is imposed on the alcohol used herein, and an alcohol that does not affect the reaction may be appropriately selected from various alcohol solvents used for general organic synthesis.

Specific examples of the alcohol include methanol, ethanol, n-propanol, i-propanol, n-butanol, i-butanol, s-butanol, t-butanol, n-pentanol, i-pentanol, s-pentanol, t-pentanol, n-hexanol, i-hexanol, s-hexanol, t-hexanol, octanol, decanol, ethylene glycol, 1,3-butanediol, and glycerin. These solvents may be used alone or in combination of two or more species.

The reaction between the ester compound of Formula (1) and the α-amino acid compound of Formula (2) may be performed at any temperature equal to or lower than the boiling point of the solvent used. In consideration of producing a target product within a short period of time at high yield, the reaction temperature is preferably 20° C. to 150° C., more preferably 40° C. to 80° C., still more preferably 65° C. to 75° C.

The reaction time cannot be univocally determined, since it varies with the reaction temperature, and the base and organic solvent species to be used. Generally, the reaction time is about 1 to 48 hours.

The reaction may be performed in such a manner that all reagents are mixed at room temperature, and then the mixture is heated to the reaction temperature. Alternatively, the reaction may be controlled by dropwise addition of a necessary reagent. The reaction may be performed in a batch, continuous, reduced-pressure, ambient-pressure, or pressurized manner. More preferably, the reaction is performed in such a manner that a base is added dropwise at ambient pressure.

After completion of the reaction, a lipidic peptide compound salt is precipitated, and thus is recovered by filtration. In consideration of ease of phase separation, the lipidic peptide compound salt is preferably an alkali metal salt.

Thereafter, the resultant lipidic peptide compound salt is dissolved in water. Preferably in water, the pH of the resultant aqueous solution is adjusted with a hydrogen halide; specifically, a hydrogen halide is added to the aqueous solution until the pH of the mixture reaches the previously calculated isoelectric point. For example, after completion of the reaction between the ester compound of Formula (1) and the α-amino acid compound of Formula (2), the precipitated lipidic peptide compound salt is filtered and redissolved in water, and then a hydrogen halide solution is added to the aqueous solution for pH adjustment. The isoelectric point, which is also referred to as “isopotential point,” is the pH value at which the formal charge becomes zero in the acid-base dissociation state of the corresponding molecule. The isoelectric point value may be calculated from the acid dissociation constant (pKa) of the molecule. For example, the isoelectric point value may be calculated on the basis of the structure of the molecule by using calculation software Calculator Plugins available from ChemAxon. Alternatively, the isoelectric point may be calculated from the actually measured zeta potential value.

The hydrogen halide used in the aforementioned neutralization operation is generally in the form of aqueous solution, in view of ease of operation. The hydrogen halide is, for example, hydrochloric acid or hydrobromic acid, and is preferably hydrochloric acid. During the pH adjustment with the hydrogen halide, when the hydrogen halide is used in an amount exceeding the amount required for the pH adjustment, a lipidic peptide hydrochloride is formed, and the recovery rate of a free form is reduced. Thus, care must be taken with the amount of the hydrogen halide used.

After the pH adjustment, a crude product of the lipidic peptide compound (free form) is recovered by, for example, filtration. If necessary, the crude product is subjected to a post-treatment process, such as washing or recrystallization, to thereby produce a purified product.

The ester compound of Formula (3) used in the present invention can be prepared through reaction between a compound of the following Formula (4) and a compound of the following Formula (5):

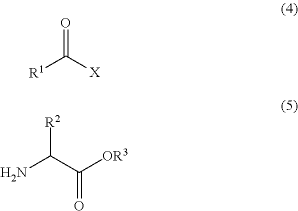

(wherein X, R¹, R², and R³ have the same meanings as defined above).

As described above, the production method of the present invention involves filtration of a lipidic peptide compound salt obtained through cooling after completion of the reaction. The lipidic peptide compound salt is redissolved in water, and then a hydrogen halide solution is added to the aqueous solution for neutralization at the isoelectric point, to thereby precipitate a target lipidic peptide compound (free form). The lipidic peptide compound can be recovered by filtration.

When the lipidic peptide compound has a gelation ability, a polar solvent (e.g., DMF) that has conventionally been used for the production of the lipidic peptide tends to cause gelation after cooling due to the action of the lipidic peptide. In this regard, the production method of the present invention is very useful in the production of the lipidic peptide, since the use of a non-polar organic solvent can prevent gelation.

Although the solution obtained through the aforementioned reaction exhibits alkalinity, the use of an aqueous hydrogen chloride solution in an amount required for pH adjustment of the solution enables completion of the pH adjustment without causing gelation, and also enables recovery of a free form. A crude crystal of the precipitated free form can be purified by any known technique such as recrystallization, to thereby produce a pure target product.

In the case where the pH adjustment is not performed, when a solution of the lipidic peptide compound salt is added dropwise to an organic solvent (i.e., poor solvent), the lipidic peptide compound salt can be reprecipitated and recovered in the form of solid.

### EXAMPLES

The present invention will next be described in more detail with reference to Synthesis Examples, Examples, and Comparative Examples, but the present invention should not be construed as being limited to the following Examples.

The commercially available reagents described below were used in Synthesis Examples and Examples, and the apparatuses described below were used for analysis and measurement of the properties of synthesized compounds.

Methanol: available from KANTO CHEMICAL CO., INC. (special grade)

Tetrahydrofuran: available from KANTO CHEMICAL CO., INC. (first grade)

i-Propanol: available from KANTO CHEMICAL CO., INC. (first grade)

Toluene: available from KANTO CHEMICAL CO., INC. (first grade)

Acetic acid: available from KANTO CHEMICAL CO., INC. (first grade)

Palmitic acid chloride: available from Aldrich (palmitoyl chloride), NOF CORPORATION (distilled palmitic acid chloride)

Glycine methyl ester hydrochloride: available from Tokyo Chemical Industry Co., Ltd.

L-Histidine: available from Tokyo Chemical Industry Co., Ltd., KYOWA HAKKO BIO Co., Ltd.

Sodium methoxide 28% methanol solution: available from Nippon Soda Co., Ltd. (liquid sodium methylate 28%), Wako Pure Chemical Industries, Ltd. (28% sodium methoxide methanol solution)

Sodium carbonate: available from JUNSEI CHEMICAL CO., LTD. (first grade), Tokuyama Corporation

Hydrochloric acid: available from KANTO CHEMICAL CO., INC. (first grade)

Acetonitrile: available from KANTO CHEMICAL CO., INC. (special grade)

NMR: JNM-ECP300 (available from JEOL Ltd.)

pH meter: available from Mettler-Toledo International Inc.

HPLC analysis conditions are as follows.

Column: Inertsil ODS-3 (available from GL sciences)

Developing solvent: MeOH/phosphate buffer (pH=2.1)=85/15 (ratio by volume)

* Preparation method for phosphate buffer (pH=2.1)

Water is added to 7.8 g (50 mmol) of sodium dihydrogenphosphate (NaH₂PO₄2H₂O) and 3.4 mL (50 mmol) of 85% phosphoric acid so that the total amount of the mixture is 1 L.

Oven temperature: 40° C.

Detection method: UV 205 nm

Flow rate: 2.0 mL/min

Injection amount: 20 μL

Retention time: N-palmitoyl-Gly-His-methyl: 5.0 min, N-palmitoyl-Gly-His: 5.5 min, N-palmitoyl-Gly: 9.3 min, N-palmitoyl-Gly-methyl: 11.2 min

### [Example 1] Synthesis of N-Palmitoyl-Gly-Methyl

A 1,000-L reaction vessel was charged with 19.0 kg (151 mol) of glycine methyl ester hydrochloride and 64 kg of water. Subsequently, 14.2 kg (134 mol) of sodium carbonate serving as a base, 96 kg of water, and 128 kg of toluene serving as an organic solvent were added to the reaction vessel, and the resultant mixture was stirred. Thereafter, 32.0 kg (116 mol) of palmitic acid chloride was added dropwise to the mixture at a reaction temperature of 25±5° C. over one hour. As a result, a white solid was precipitated to form a slurry. The slurry was stirred at 25° C. for three hours, and then 320 kg of 10% salt water was added to the slurry, followed by a phase separation operation at 60° C. Subsequently, 256 kg of toluene was added to the resultant organic phase, and the mixture was subjected to azeotropic dehydration, to thereby produce 295.7 kg of a toluene solution of N-palmitoyl-Gly-methyl (yield: 94%).

*NMR analysis was performed after removal of the solvent.

¹H-NMR (300 MHz, MeOH-d₄, δ ppm): 3.97 (2H, s), 3.71 (3H, s), 2.23 (2H, t, J=7.4 Hz), 1.61 (2H, m), 1.28 (24H, m), 0.89 (3H, t, J=6.8 Hz)

MS (ESI) m/z: 327.78 (Mc)

Melting point: 78.1° C.

### [Example 2] Synthesis of N-Palmitoyl-Gly-His Free Form

A 500-L reaction vessel was charged with 17.0 kg (110 mol) of histidine and 716 kg of toluene, and 20.0 kg (104 mol) of 28% sodium methoxide methanol solution serving as a base was added dropwise to the reaction vessel. After azeotropic dehydration, the toluene solution of N-palmitoyl-Gly-methyl produced in Example 1 and 14.3 kg of methanol were added to the resultant mixture, and the mixture was heated to 70° C. Thereafter, 7.4 kg (38 mol) of 28% sodium methoxide methanol solution serving as a base was added dropwise to the mixture, and the resultant mixture was stirred at about 70° C. for 16 hours. After completion of the reaction, the resultant slurry was cooled and subjected to filtration, and the resultant N-palmitoyl-Gly-His sodium salt was dried at 40° C. under reduced pressure.

The resultant N-palmitoyl-Gly-His sodium salt was dissolved in 30.7 kg of toluene and 1,181.7 kg of water, and the solution was heated to 60° C. The pH of the solution was adjusted to 4.5 with 35% hydrochloric acid for neutralization, to thereby precipitate a crude crystal of N-palmitoyl-Gly-His free form. The resultant product was cooled and then subjected to filtration, followed by drying at 80° C. under reduced pressure, to thereby prepare 48.2 kg of a crude crystal.

### Example 3

To the dried crude crystal were added 408.0 kg of toluene and 244.8 kg of methanol, and the resultant mixture was heated to 60° C. for dissolution. Thereafter, 163.2 kg of tetrahydrofuran was added to the solution, and the resultant mixture was cooled. The precipitated crystal was filtered, and dried at 80° C. under reduced pressure, to thereby produce 37.9 g of a white crystal of N-palmitoyl-Gly-His free form (purity: 99.3%, yield: 95.7%).

¹H-NMR (300 MHz, DMSO-d₆, δ ppm): 8.12 (1H, d, J=7.8 Hz), 8.06 (1H, t, J=5.7 Hz), 7.56 (1H, s), 6.81 (1H, s), 4.38 (1H, q, J=7.8 Hz), 3.69 (2H, dd, J=5.7 Hz and J=10.2 Hz), 2.89 (2H, m), 2.20 (2H, t, J=6.9 Hz), 1.48 (2H, m), 1.23 (24H, s), 0.85 (3H, t, J=7.2 Hz)

MS (EI) m/z: 451.43 (M⁺+1)

1. A production method for a lipidic peptide compound of the following Formula (3): 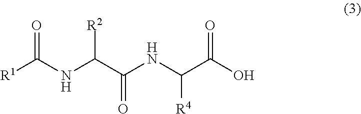
(wherein R¹, R², and R⁴ have the meanings as defined below) or a pharmaceutically usable salt thereof, the production method being characterized by comprising:
a mixing step of mixing a non-polar solvent containing an ester compound of the following Formula (1): 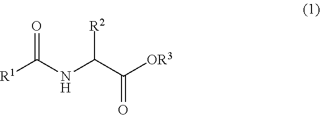
(wherein R¹ is a C₉₋₂₃ aliphatic group; R² is a hydrogen atom or a C₁₋₄ alkyl group possibly having a branched chain having a carbon atom number of 1 or 2; and R³ is a C₁₋₆ alkyl group, a C₁₋₆ haloalkyl group, a C₁₋₆ hydroxyalkyl group, or an aryl group substitutable with a C₁₋₆ alkyl group) with a non-polar organic solvent containing an α-amino acid compound of the following Formula (2): 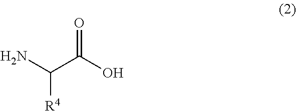
(wherein R⁴ is a —(CH₂)ₙ—X group; n is a number of 1 to 4; and X is an amino group, a guanidino group, a —CONH₂ group, or a 5-membered or 6-membered ring or condensed heterocyclic ring composed of 5-membered and 6-membered rings possibly having one to three nitrogen atoms) and a base.

2. The production method according to claim 1, characterized in that the non-polar solvent contains a non-polar organic solvent and an alcohol.

3. The production method according to claim 1, wherein, in the aforementioned formula, n is a number of 1 to 4, and X is an amino group, a guanidino group, or a —CONH₂ group; or n is 1, and X is a pyrrole group, an imidazole group, a pyrazole group, or an imidazole group.

4. The production method according to claim 1, wherein, in the aforementioned formula, R¹ is a Cu-21 aliphatic group having a linear or branched structure and possibly having zero to two unsaturated bonds.

5. The production method according to claim 1, wherein, in the aforementioned formula, R² is a hydrogen atom or a C₁₋₃ alkyl group possibly having a branched chain having a carbon atom number of 1.

6. The production method according to claim 1, wherein, in the aforementioned formula, R² is a hydrogen atom, a methyl group, an ethyl group, an n-propyl group, an isopropyl group, an n-butyl group, an isobutyl group, a sec-butyl group, or a tert-butyl group, and R⁴ is an aminomethyl group, an aminoethyl group, a 3-aminopropyl group, a 4-aminobutyl group, a carbamoylmethyl group, a 2-carbamoylethyl group, a 3-carbamoylbutyl group, a 2-guanidinoethyl group, a 3-guanidinopropyl group, a pyrrolemethyl group, an imidazolemethyl group, a pyrazolemethyl group, or a 3-indolemethyl group.

7. The production method according to claim 6, wherein, in the aforementioned formula, R² is a hydrogen atom, a methyl group, an isopropyl group, an isobutyl group, or a sec-butyl group, and R⁴ is a 4-aminobutyl group, a carbamoylmethyl group, a 2-carbamoylethyl group, a 3-guanidinopropyl group, an imidazolemethyl group, or a 3-indolemethyl group.

8. The production method according to claim 1, wherein, in the aforementioned formula, R³ is a methyl group or an ethyl group.

9. The production method according to any one of claims 1 to 8, wherein the base is at least one selected from among an alkali metal, an alkali metal inorganic acid salt, an alkali metal hydroxide, an alkali metal alkoxide, an alicyclic amine, or an alcohol solution of any of these, or an alcohol dispersion of any of these.

10. The production method according to claim 9, wherein the base is at least one selected from among metallic sodium, metallic potassium, sodium carbonate, potassium carbonate, potassium phosphate, sodium phosphate, sodium hydroxide, potassium hydroxide, sodium methoxide, sodium ethoxide, potassium methoxide, potassium ethoxide, t-butoxypotassium, 1,8-diazabicyclo[5.4.0]-7-undecene, 1,5-diazabicyclo[4.3.0]-5-nonene, or an alcohol solution of any of these, or an alcohol dispersion of any of these.

11. The production method according to claim 10, wherein the base is sodium methoxide, or a methanol solution or methanol dispersion thereof.

12. The production method according to any one of claims 1 to 11, wherein the non-polar organic solvent is at least one selected from the group consisting of an aromatic compound, a saturated aliphatic compound, and an unsaturated aliphatic compound.

13. The production method according to claim 12, wherein the non-polar organic solvent is at least one selected from the group consisting of toluene, xylene, o-dichlorobenzene, pentane, hexane, heptane, octane, cyclopentane, cyclohexane, methylcyclohexane, cycloheptane, and 1-hexene.

14. The production method according to claim 2, wherein the non-polar solvent contains toluene and methanol or ethanol.

15. The production method according to any one of claims 1 to 14, wherein the reaction between the ester compound of Formula (1) and the α-amino acid compound of Formula (2) is performed at a reaction temperature of 70±5° C.

16. The production method according to any one of claims 1 to 15, wherein the method comprises a precipitation step of treating a product produced through the reaction between the ester compound of Formula (1) and the α-amino acid compound of Formula (2) with a hydrogen halide so that the pH of the product is adjusted to the isoelectric point of the lipidic peptide compound, thereby precipitating the lipidic peptide.

17. The production method according to claim 16, wherein the precipitation step is performed at 40° C. to 70° C.

18. A production method for a lipidic peptide compound of the following Formula (3): 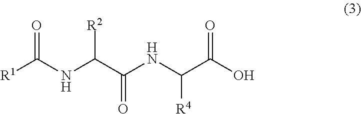
(wherein R¹, R², and R⁴ have the meanings as defined below) or a pharmaceutically usable salt thereof, the production method being characterized by comprising:
a step of reacting a compound of the following Formula (4): 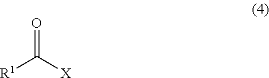
with a compound of the following Formula (5): 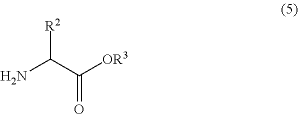
(wherein R² is a hydrogen atom or a C₁₋₄ alkyl group possibly having a branched chain having a carbon atom number of 1 or 2; and R³ is a C₁₋₆ alkyl group, a C₁₋₆ haloalkyl group, a C₁₋₆ hydroxyalkyl group, or an aryl group substitutable with a C₁₋₆ alkyl group), thereby producing an ester compound of the following Formula (1): 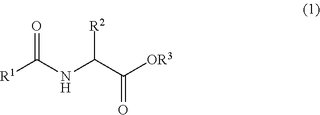
and
a mixing step of mixing a non-polar solvent containing the ester compound of Formula (1) with a non-polar organic solvent containing an α-amino acid compound of the following Formula (2): 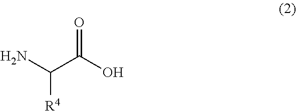
(wherein R⁴ is a hydrogen atom, a C₁₋₇ alkyl group possibly having a branched chain having a carbon atom number of 1 to 3, a phenylmethyl group, a phenylethyl group, or a —(CH₂)ₙ—X group; n is a number of 1 to 4; and X is an amino group, a guanidino group, a —CONH₂ group, or a 5-membered or 6-membered ring or condensed heterocyclic ring composed of 5-membered and 6-membered rings possibly having one to three nitrogen atoms) and a base.
